(19) United States
(12) Patent Application Publication
(10) Pub. No.: US 2009/0318791 A1
(43) Pub. Date: Dec. 24, 2009

(54) Perfusion Device with Compensation of Medical Infusion During Wear-Time

(75) Inventor: Peter Kaastrup, Maeloev (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 12/305,688

(22) PCT Filed: Jun. 15, 2007
(86) PCT No.: PCT/EP2007/055950

§ 371 Date: Jan. 22, 2009

(30) Foreign Application Priority Data
Jun. 30, 2006 (EP) 06116397.8

(51) Int. Cl. A61M 5/168; A61M 5/31; A61B 5/00

(52) U.S. Cl. 600/365; 604/93.01; 604/67

(57) ABSTRACT

A medical action device is adapted to cooperate with an object to be inserted into a body and to cooperate with a timing device, able to start timing from the moment the object is inserted. This information is fed to a control unit whereby the control unit gets an input which can be used to compensate one or more parameters of the medical action as a function of the increased blood flow in the area around the inserted object over time due to the inflammation reaction.

The invention relates to a medical device enabled to perform a medical action. The medical device can be used in combination with an object inserted into a body. invention is to adapt the medical device to compensate one or more parameters of the medical action in relation to the time elapsed from when the object was inserted into the body.

### BACKGROUND OF THE INVENTION

In diabetes treatment a major concern is to maintain an acceptable, close to natural blood glucose level of a patient around the clock. Injecting insulin in a one or a number of discrete doses on a daily or weekly basis will normally result in blood stream peaks in insulin levels, as these discrete doses are to compensate for many small or big fluctuations in a patients blood glucose level due to meals, exercise etc.

Variation of the blood glucose concentration outside the natural interval on short term can cause acute crisis and on long term can cause diabetes complications such as heart disease, stroke, blindness, amputations and renal failure. Therefore it is desirable with a close-to-continuous administration of insulin to the patient.

A way of obtaining a high number of discrete administrations of insulin and thereby getting a blood glucose control with a profile closer the natural, is by the use of insulin pumps, administering the insulin through a constantly inserted catheter. Because the catheter is constantly inserted, the user will not feel the discomfort of having to insert a needle every time a dose is injected. Therefore the much higher frequency of injections which permits for better blood glucose level control, is not associated with a higher level of immediate discomfort for the user. Also the absorption of insulin from a pump site is very efficient and predictable. Insulin pumps use only a single short-acting type of insulin, having a much more efficient 3% day-to-day absorption variance than the basal insulin types. Unlike with multiple daily injections, pump therapy uses a single infusion site, usually in the abdomen. Multiple injection sites can result in unpredictable absorption during exercise and increase the risk of hypoglycemia. because the absorption of insulin from a pump site is so efficient and predictable, patients can decrease their daily insulin dose, causing more precise control. Information relevant to this subject can be seen in: “EMERGENCY MEDICINE®: The Practice Journal for Emergency and Urgent Care, cover article Sep. 15, 2002: Insulin Pump Therapy: What You Need to Know. By Jeff Unger, MD, and Alan O. Mar-cus, MD” and “Weissberg-Benchell J, ntisdel-Lomaglio J, Seshadri R. Insulin Pump Therapy: A meta-analysis. Diabetes care 2003; 26(4):1079-1087”

As the pump therapy has the advantage of more precise and predictable blood-glucose-level control, off course it is desirable to minimize any source of error disturbing the predictability of the absorption level.

Normal tissue is disturbed when a needle inserter with a sensor or an infusion needle is introduced into the tissue. Penetration of the outermost though skin needs sharp or cutting edges of the inserter or needle. When the penetration goes further into the much more soft subcutaneous tissue target, these cutting edges lead to unwanted bleeding and tissue wounds together with an over time arising inflammatory response and foreign body reaction against the penetrating materials.

In addition to the discomfort, such tissue wounds and inflammation also cause changes in the local blood flow. Wear time dependent changes in the local blood flow disturbs the function/precision of the sensor or disturbs the predictability of infusion drug flow from the local infusion site to the whole body. As result it causes higher frequency of unwanted hypoglycemia, or unwanted too high blood glucose level and the associated unwanted serious side effects of this to the health. References concerning the importance of blood-flow to absorption are: “Vora, A Burch, J R Peters and D R Owens: Relationship between absorption of radiolabeled soluble insulin, subcutaneous blood flow, and anthropometry, Diabetes Care, Vol 15, Issue 11 1484-1493, 1992” and “Vora J P, Burch A, Peters J R, Owens D R.: Absorption of radiolabelled soluble insulin in type 1 (insulin-dependent) diabetes: influence of subcutaneous blood flow and anthropometry. Dia-bet Med. 1993 October; 10(8):736-43”.

The problem of the change of blood-flow over time in the local area around an object inserted into a body can relate to any situations where a prolonged insertion is needed. Without restricting the invention to the following examples, relevant fields to be mentioned are: insulin pumps adapted to cooperate with an object inserted into the body, sensors inserted into the body, measuring physiological parameters such as blood glucose level and infusion devices. A reference to this subject is WO 99/32174.

Implanted blood glucose sensors are discussed in: Kvist, P. H., et al. “Evaluation of subcutaneously-implanted glucose sensors for continuous glucose measurements in hyperglycemic pigs.” In Vivo 20.2 (2006): 195-204. And known methods of measuring blood flow are disclosed in: Bulow J. Measurement of adipose tissue blood flow. Methods Mol Biol 2001; 155:281-293 and Bulow J, Jelnes R, Astrup A, Madsen J, Vilmann P. Tissue/blood partition coefficients for xenon in various adipose tissue depots in man. SCAND J CLIN LAB INVEST 1987; 47(1):1-3. Further the measurement of inflammation in the area of an inserted object in a body is discussed in WO2004060455A1, however here the determination of inflammation rely on real time measurements

In view of the above, one of the objectives of the present invention is to provide a technical solution to compensate for the wear time dependent changes in the local blood flow around an object inserted into a body

It is a further objective of the present invention to provide a medical device to perform a medical action and adapted to be used in combination with a member to be inserted into a body which is adapted to change one or more parameters of the action performed by the device, for instance the timing or flow rate of an infusion, or the timing of a measurement.

A further objective is also to ensure ease of handling by the user, for instance by incorporating an algorithm into a controller of the medical device, whereby a given time wear compensation can be performed without direct control by the user.

Still a further objective of the present invention is to provide an insulin expelling device which gives better blood glucose level control providing less late stage diabetic complications, less frequency of unwanted hypoglycemia episodes than yet known devices.

### SUMMARY OF THE INVENTION

In the disclosure of the present invention, embodiments and aspects will be described which will address one or more of the above objectives or which will address objectives apparent from the below disclosure as well as from the description of exemplary embodiments.

In a first aspect of the invention, a medical drug expelling device, more specific a portable drug delivery device for delivering a drug to a patient comprise a reservoir adapted to contain a liquid drug and having an outlet in fluid communication with a hollow infusion needle, as well as expelling means for expelling a drug out of the reservoir and through the skin of a body via the hollow needle. The devices comprise a mounting surface adapted for application to the skin of a body by adhesive means, and a transcutaneous device adapted to be inserted through the skin of the body, e.g. a needle or a soft cannula, a micro needle array, a traditional infusion set or non-invasive transdermal means, projecting from or arranged on a lower surface of a skin-mountable device in a situation of use. The needle or the soft cannula may be insertable after the device has been arranged on the skin.

The drug reservoirs used for the device may be in the form of a “hard” reservoir (e.g. a cylinder-piston reservoir) or a flexible reservoir. The “hard” reservoir provides inherently good protection against accidental compression of the reservoir from the outside, thereby reducing the risk of unintended expelling of drug from the device and into the body when subjected to excessive forces, e.g. the patient carrying a skin-mounted infusion device may stumble or walk into a hard object, or the device may be hit by an object. Depending on the construction of the device, a flexible reservoir may be arranged “downstream” of the expelling means, e.g. as for a gas generating pump, or “upstream” of the expelling means, e.g. as for a suction pump. The pump assembly may further comprise an actuator for actuating the pump, or it may alternatively be adapted to cooperate with an external pump actuator. For example, the pump assembly may be provided in combination with a prefilled reservoir as a disposable unit, whereas the pump actuator may be incorporated in a durable unit adapted to be coupled to the disposable unit. The durable unit may also comprise an energy source and control electronics for operating the pump.

Essential to the present invention is that the medical device, whatever the function is, as long at it is cooperating with an object inserted into a body, and is cooperating with a timer. The timer will send input to the control unit of the medical device, the input being a signal or a value corresponding the time elapsed since the object was inserted into the body. This signal enables the control unit to compensate for the changed flow of the blood stream in the area around the inserted object.

### DESCRIPTION OF THE DRAWINGS

In the following the invention will be further described with references to the drawings, wherein

FIG. 1 shows a table illustrating the dependency between elapsed time and blood flow in the area of an inserted object.

FIG. 2 shows a table illustrating the dependency between elapsed time and blood flow in the area of an inserted object for a further number of points in time since insertion and specific to a number of subjects.

### DESCRIPTION OF EXEMPLARY EMBODIMENTS

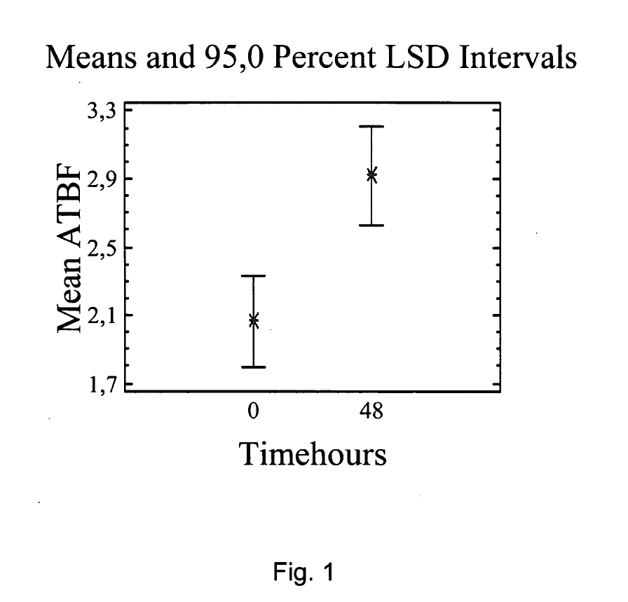

FIG. 1 depicts how the blood flow changes over time in the area around a soft catheter inserted in a body for expelling a drug. As can be seen, there is an increase of approximately 40% in subcutaneous Adipose tissue Blood Flow (ATBF) after two days. At the time immediately after the catheter has been inserted the ATBF has a mean value of approximately index 2.1. After the catheter has been inserted for 48 hours, the ATBF index value is 2.9—a difference corresponding a raise in ATBF of 38%. Also shown on the figure are the intervals around the mean ATBF values corresponding the 95% statistical confidence level.

The medical device of the invention is equipped with a timer for registration of the time where the catheter was inserted in a body. This information is delivered to a control unit of the medical device, which control unit is also programmed with a decision support software algorithm which is used by the control unit, when calculating the compensating action correlating the elapsed time from the insertion of the catheter. The more time has elapsed, the greater the compensation.

The registered time elapsed is a single parameter, whereas the compensating action correlating this elapsed time can be a product of many, more complicated factors, either calculated or loaded into the control device. The factors can be individually dependant and estimated by a health care professional, they can be dependant of the location of the catheter on the body, they can be constants, they can be dependant of the drug type, the catheter type, the medical device type, this list by no means being complete.

Presuming the liquid drug to be injected into the body is insulin for diabetes treatment, the actual algorithm for calculating the relation between elapsed time since inserting an object into a body and absorption of the insulin into the blood stream can be a further improvement of the Berger model for calculating the size of a doze to be injected. The Berger model has a factor which relates to the time for absorption of insulin into the blood, the berger model being as follows:

$$\frac{d A ( t )}{d t} = \frac{s \cdot {t}^{s - 1} \cdot {T}_{50}^{s}}{{( {T}_{50}^{s} + {t}^{s} )}^{2}} \cdot dose - k \cdot A ( t )$$

T₅₀: The half time for absorption of insulin from depot to plasma. T₅₀=a·dose.
a: Characterizes the dose dependency of the absorption time.
k: The first-order elimination constant.

As T₅₀ in Bergers model is time dependent, T₅₀ is the half life time for absorption of insulin into the blood, T₅₀ being a·dose, and this formula can be improved to compensate for the relation between insulin absorption in the blood stream depending on time. This can be done by adding a wear time factor, wt(Ct), dependent on catheter (or other object inserted) wear time Ct such that T₅₀ being a·dose·wt(Ct).

On the basis of the values in FIG. 1, it is determined that a elapsed time of 48 hours from the time a member was inserted into a body, the local blood flow in that body area increases with 38%. If the inserted member is a needle or a catheter injecting og infusing insulin, it is known from Weinzimer 2005 that an increase in blood flow of 38% will increase absorption rate of insulin to blood with 25%. Wt(Ct) can then be determined and T₅₀ at 48 hours is then calculated as 1/1.25 and a linear extrapolation for other wear time factors is performed.

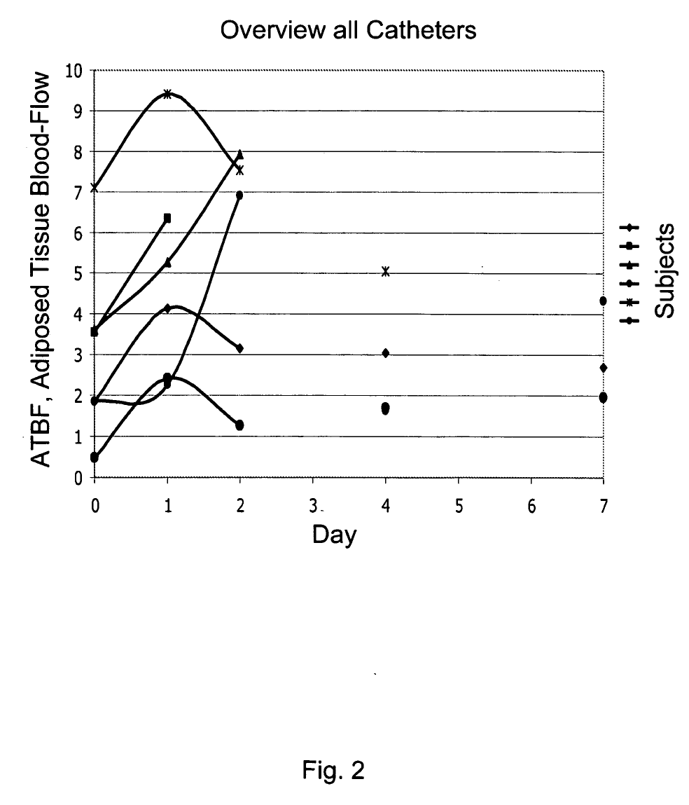

On the basis of the values in FIG. 2, having values for Adiposed Tissue Blood-Flow ATBF, for a further number of points in time from the start time where the insertion member was inserted into a body, a further number of wear time factors can be determined, relating to the points or time periods accordingly. Thus as many wear time factors as wanted can be calculated, also on a specific personal basis varying from person to person. It can be advantageous to calculate a relatively small number of wear times, for instance a first wear time factor can be determined for a first time period, relating to the first response to the inserted member, on FIG. 2 it can be seen that this period could estimated to an interval from insertion time until 24-48 hours. A second wear time factor can be determined for a second time period ranging from 24 or 48 hours until 48-72 hours after insertion, and even a third wear time factor can be determined for a third time period ranging from 48-72 hours until the time where the insertion member is again retracted.

On the basis of the timer input (wear time data), the algorithm and the programmed factors, an output from the control unit is calculated. In the exemplary case of an insulin expelling device, the output can be a variety of the following, the list of actions not being complete:
The bolus start time can be compensated, more specific, the timing can be delayed the longer time has elapsed since the catheter was inserted.
The pump speed can be changed, so as to keep a uniform insulin bolus pharmacokinetic profile in the blood stream. More specific, as the wear time increases, the bolus pump speed is decreased to compensate for the higher blood flow around the catheter.
Specific for an insulin piston pump, the piston displacement can be decreased as wear time is increasing, combined with an increased number of discrete displacements to achieve the desired amount of bolus insulin.
Likewise specific for an insulin piston pump, the piston stroke frequency can be decreased as the wear time increases, combined with an extended time span to achieve the desired amount of bolus insulin.
Further relevant for an insulin membrane pump, the membrane displacement stroke frequency can be decreased as wear time is increasing
A blood glucose output value can be delayed according to the time elapsed since insertion of the insertion member

General is that a number of wear time factors can be determined and preprogrammed into the controller in a look-up table and then directly used in the algorithm to calculate dose size, flow or delay, or the wear time factors be determined by an algorithm of the device on the basis of the output of a timer in the device corresponding to the time elapsed since the sensor, needle or catheter was inserted into the body, and then uses to calculate dose size, speed or delay. The relation between the blood-flow and the insertion time can be determined in advance in general, person-specific or even insertion area-specific, thus, the determination of the wear time factor does not rely on any real-time blood flow measurements.

### FEATURES OF THE INVENTION

1. A medical device adapted to perform a medical action, adapted to be used in combination with a member to be inserted into a body,

characterized in, said medical device is adapted to change one or more parameters of the medical action according to the time elapsed from the insertion of said member into the body, thereby compensating for the change of blood-flow over time in the body area of the inserted member due to inflammation.

2. A medical device according to clause 1,

characterized in, the medical device comprising a controller, said controller comprises an algorithm adapted to postpone the timing of said medical action according to the time elapsed from insertion of said member into the body, thereby compensating for the acceleration of blood-flow over time in the body area of the inserted member due to inflammation.

3. A medical device according to any of the clauses 1 or 2,

characterized in, the medical action is an injection, and infusion or a monitoring of a physiological parameter.

4. A medical device according to clause 3,

characterized in, the medical device comprising a controller, said controller comprises an algorithm adapted to decrease the infusion- or injection flow rate according to the time elapsed from insertion of said member into the body, thereby compensating for the acceleration of blood-flow over time in the body area of the inserted member due to inflammation.

5. A medical device according to clause 2, 3 or 4,

characterized in, said algorithm comprising a wear time factor, wt(Ct) dependent of the wear time of the inserted member.

6. A medical device according to clause 5,

characterized in, said wear time factor, wt(Ct), being the following values: 1 (0 hours), 0.95 (12 hours), 0.9 (24 hours), 0.85 (36 hours), 0.8 (48 hours), 0.75 (60 hours), 0.7 (72 hours).

7. A medical device according to any of the preceding clauses,

characterized in, said medical device is an expelling device adapted to cooperate with a needle or a catheter inserted into a body.

8. A medical device according to clause 7,

characterized in, said medical device is an expelling device, said expelling device is adapted to reduce the flow rate by reducing the stroke volume or the stroke frequency.

9. A medical device according to any of the preceding clauses,

characterized in, the device comprising a timer for measuring the time elapsed from insertion of said member into the body.

10. A portable medical device adapted to perform a medical action, the medical action comprising an injection, an infusion or a monitoring of a physiological parameter, said device is in communication or has a fluid connection with a member to be inserted into a body,
said device comprising a timer adapted to produce an output corresponding to the time elapsed from the insertion of said member into the body, and a controller, the controller comprising an algorithm or a look-up table,

characterized in, said controller is adapted to determine a wear time factor on the basis of the timer output and said algorithm or said look-up table, said wear time factor is applied by said controller to postpone said medical action or adjust one or more parameters of the medical action according to the time elapsed from the insertion of said member into the body.

11. A medical device according to clause 10.

characterized in, said postponement or adjustment are discrete constant values corresponding to discrete periods of time starting at the time said member was inserted into the body, said discrete constant values are pre-programmed into the controller.

12. A medical device according to clause 10 or 11,

characterized in, said portable device is a drug injection or infusion device and said controller is adapted to decrease the infusion flow rate or injection dose size according to the time elapsed from insertion of said member into the body in a first time period, to increase the infusion flow rate or injection dose size in a second time period, and to decrease the infusion flow rate or injection dose size in a third time period.

13. A medical device according to clause 10 or 11,

characterized in, said portable device is adapted to monitor blood glucose and that the blood glucose output value is delayed for a first time value during a first time period starting at the time said member was inserted into the body, the glucose output value is delayed for a second time value being smaller than the first time value and the glucose output value is delayed for a third time value being larger than the second time value.

14. A medical device according to any of the preceding clauses,

characterized in, said wear time factor, wt(Ct), being the following values: 1 (0 hours), 0.95 (12 hours), 0.9 (24 hours), 0.85 (36 hours), 0.8 (48 hours), 0.75 (60 hours), 0.7 (72 hours).

15. A medical device according to any of the clauses

characterized in, said wear time factor, wt(Ct) being the following values: 1 (0 hours), in the interval 0.5-0.9 (0-24) hours and in the interval 0.7-0.95 (24 hours-end time of member insertion).

16. A medical device according to any of the clauses

characterized in, said wear time factor, wt(Ct) being the following values: 1 (0 hours), in the interval 0.5-0.9 (0-48) hours and in the interval 0.7-0.95 (48 hours-end time of member insertion).

17. A medical device according to any of the preceding clauses 10-12 and 14-16,

characterized in, said medical device is an expelling device, said expelling device is adapted to reduce the flow rate by reducing the stroke volume or the stroke frequency.

18. A medical device according to any of the preceding clauses,

characterized in, said medical device is an insulin injection or insulin infusion device.

1. A portable medical device adapted to perform a medical action, the medical action comprising an injection, an infusion or a monitoring of a physiological parameter, said device is in communication or has a fluid connection with a member to be inserted into a body,
said device comprising a timer adapted to produce an output corresponding to the time elapsed from the insertion of said member into the body,
and a controller, the controller comprising an algorithm or a look-up table,
wherein said controller is adapted to determine a wear time factor on the basis of the timer output and said algorithm or said look-up table, said wear time factor is applied by said controller to postpone said medical action or adjust one or more parameters of the medical action according to the time elapsed from the insertion of said member into the body.

2. A medical device according to claim 1,
wherein said postponement or adjustment are discrete constant values corresponding to discrete periods of time starting at the time said member was inserted into the body, said discrete constant values are pre-programmed into the controller.

3. A medical device according to claim 1,
wherein said portable device is a drug injection or infusion device and said controller is adapted to decrease the infusion flow rate or injection dose size according to the time elapsed from insertion of said member into the body in a first time period, to increase the infusion flow rate or injection dose size in a second time period, and to decrease the infusion flow rate or injection dose size in a third time period.

4. A medical device according to claim 1,
wherein said portable device is adapted to monitor blood glucose and that the blood glucose output value is delayed for a first time value during a first time period starting at the time said member was inserted into the body, the glucose output value is delayed for a second time value being smaller than the first time value and the glucose output value is delayed for a third time value being larger than the second time value.

5. A medical device according to claim 1,
wherein said wear time factor, wt(Ct), being the following values: 1 (0 hours), 0.95 (12 hours), 0.9 (24 hours), 0.85 (36 hours), 0.8 (48 hours), 0.75 (60 hours), 0.7 (72 hours).

6. A medical device according to claim 1,
wherein said wear time factor, wt(Ct) being the following values: 1 (0 hours), in the interval 0.5-0.9 (0-24) hours and in the interval 0.7-0.95 (24 hours-end time of member insertion).

7. A medical device according to claim 1,
wherein said wear time factor, wt(Ct) being the following values: 1 (0 hours), in the interval 0.5-0.9 (0-48) hours and in the interval 0.7-0.95 (48 hours-end time of member insertion).

8. A medical device according to claim 1,
wherein said medical device is an expelling device, said expelling device is adapted to reduce the flow rate by reducing the stroke volume or the stroke frequency.

9. A medical device according to claim 1,
wherein said medical device is an insulin injection or insulin infusion device.

10. A medical device according to claim 5, wherein said medical device is an expelling device, said expelling device is adapted to reduce the flow rate by reducing the stroke volume or the stroke frequency.
